(19) United States
(12) Patent Application Publication
(10) Pub. No.: US 2009/0234236 A1
(43) Pub. Date: Sep. 17, 2009

(54) NERVE BLOOD FLOW MODULATION FOR IMAGING NERVES

(75) Inventors: Stephen Johnson LOMNES, Philadelphia, PA (US); Cristina Abucay TAN HEHIR, Niskayuna, NY (US); Deborah Stutz LEE, Niskayuna, NY (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: General Electric Company, Schenectady, NY (US)

(21) Appl. No.: 12/048,331

(22) Filed: Mar. 14, 2008

(51) Int. Cl. A61B 5/02

(52) U.S. Cl. 600/504

(57) ABSTRACT

A method of visualizing nerves by observing the hemodynamic response of the blood flow comprising: acquiring a pre-stimulus image of a target tissue; providing a stimulus to the target tissue; introducing a time delay between the stimulus and a post-stimulus image; capturing the post-stimulus image of the target tissue; and producing a processed image based on a comparison between the pre-stimulus image and the post-stimulus image. Also described is a system for evaluating the hemodynamic response of blood flow comprising producing a processed image based on a comparison between the pre-stimulus image and the post-stimulus image.

### BACKGROUND OF THIL INVENTION

1. Field of the Invention

The subject matter disclosed herein relates generally to the area of optical imaging, and more particularly to a method and system of imaging nerves using a stimulus to modulate nerve blood flow.

2. Description of the Related Art

The identification of nerves in a surgical field is challenging and often iatrogenic injury occurs in nerve structures. Often this results in undesirable complications as a result of surgery such as numbness, impaired motor function, or impotence. To avoid or reduce damage to functional neuronal tissue, numerous techniques have been developed for neuronal detection and intraoperation assessment.

Current intraoperative techniques for localizing neuronal function during neurosurgery include electroencephalography (EEC) and electrocorticography (ECoG). Such techniques provide a direct measure of brain electrical activity, in contrast to positron emission tomography (PET) scans which look at blood flow and metabolic changes in tissue and computed tomography (CT) scans which look at tissue density differences, and which are typically used in preoperative evaluation of a patient. Additional techniques include, among others, spectroscopic techniques (e.g., electron microscopy and x-ray diffraction), phase-contrast microtomography (p-mCT), magnetic resonance imaging (MRI), ultra-sound, and other physiological studies measuring intrinsic fluorescence, use of voltage-sensitive dyes, and reflection measurements of tissue in response to electrical or metabolic activity. See, e.g., Blasdel, G. G. and Salama, G., “Voltage Sensitive Dyes Reveal a Modular Organization Monkey Striate Cortex,” Nature 321:579-585, 1986); Grinvald, A., et al., “Functional Architecture of Cortex Revealed by Optical Imaging of Intrinsic Signals,” Nature 324:361-364, 1986); Ts'o, D. Y., et al. “Functional Organization of Primate Visual Cortex Revealed by High Resolution Optical imaging,” Science 249:417-420 (1990). Numerous references describe optically imaging neuronal tissue and other types of tissue using these and other techniques. See, e.g., U.S. Pat. Nos. 5,215,095; 5,438,989; 5,465,718; 6,233,480; 6,564,079; US. Pub. Nos. 2003/0152962; 2005/026754; 2007/0122344; and WO/2002100247, among others.

A common method of intraoperative localization of neuronal function during neurosurgery is direct electrical stimulation with a stimulating electrode. Neuronal activity can be both stimulated and observed on a millisecond time scale utilizing electrical measurements and these activities can be correlated with coupled changes in the hemodynamic delivery of glucose and oxygen to local neuronal tissues through the blood vessels. If a stimulus is presented to the central nervous system, two kinds of evoked responses are generated. The first appears within a millisecond time scale (5 to 500 ms) and is an electrical response that can be evaluated in the electroencephalogram. The second evoked response appears within a few seconds and corresponds to an increase in cerebral blood flow to the region of active neuronal tissue. This second response can be evaluated by several methods including direct observation of the delivery of fluorescent dyes, measurement of the blood oxygen level-dependent signal in functional MRI, and measurement of hemoglobin signals in near-infrared (NIR) spectrophotometry. See, generally, Y. Tong et al, “Fast optical signals in the peripheral nervous system,” J. Biomed. Optics 11, 044014 (2006); see also, e.g., A. F. Cannestra et al., “Refractory periods observed by intrinsic signal and fluorescent dye imaging,” J. Neurophysiol. 80, 1522-1532 (1998); J. W. Belliveau et al, “Functional mapping of the human visual cortex by magnetic resonance imaging,” Science 254, 716-719 (1991); Y. Hoshi et alt, “Dynamic multichannel near-infrared optical imaging of human brain activity,” J. Appl. Physiol. 75, 1842-1846 (1993).

A variety of dyes useful for medical imaging have also been described, including fluorescent dyes, colorimetric dyes and radio opaque dyes. See, e.g., U.S. Pat. Nos. 5,699,798; 5,279,298; 6,351,663. Some dyes can serve both an imaging function and a therapeutic function. See, e.g., U.S. Pat. No. 6,840,933. Some specific neuronal imaging agents have been used to visualize tissue in the central nervous system.

The potential application of optical techniques to the evaluation and measurement of neurovascular coupling is significant because of the potential for sensing changes in neuronal tissue on both millisecond and second time scales. Optical methods are sensitive to interactions with biological tissues at varying temporal and spatial scales and thus can image both structural and physiological changes. Optical methods have proven to be a very useful for monitoring neuronal responses in both the central and the peripheral nervous system. See, e.g., Y. Tong et at, “Fast optical signals in the peripheral nervous system,” J. Biomed. Optics 11, 044014 (2006); K. Sato et al., “Intraoperative intrinsic optical imaging of neuronal activity from subdivisions of the human primary somatosensory cortex,” Cerebral Cortex 12, 269-290 (2002); M. M. Haglund et al., “Optical imaging of epileptiform and functional activity in human cerebral cortex, Nature 358, 668-671 (1992); D. Y. Ts'o et al., “Functional organization of primate visual cortex revealed by high resolution optical imaging,” Science 249, 417-420 (1990).

Tong et al. studied the near-infrared optical response to electrical stimulation of peripheral nerves. The authors stimulated the sural nerve of six subjects with transcutaneous electrical pulses and evaluated optical changes that peaked 60 to 160 ms after the electrical stimulus. On the basis of the strong wavelength dependence of these fast optical signals, the authors posited a rapid hemodynamic response to electrical nerve activation. These findings and others strongly suggest that the peripheral nervous system undergoes neurovascular coupling.

A need exists in the field for improved systems and methods for detecting and imaging neuronal tissue. Taking into account the observations noted above, we have determined that digital imaging systems may be employed to identify nerves in and around a surgical site. Digital imaging systems have become increasingly useful in a variety of fields. For example, in the medical diagnostics field, image data may be acquired through various modality systems, including MRI systems, computed tomography (CT) systems, x-ray systems, ultrasound systems, and so forth. Depending upon the imaging modality, the image data may be further processed, filtered, enhanced, scaled, and so forth to reduce noise and to render more visible particular features of interest. The resulting image may be viewed by a user, such as on a computer monitor or similar display, often referred to as softcopy, or may be output as hard copy, such as on a paper or similar support, or photographic film.

We describe herein novel systems and methods for visualizing neuronal tissue by providing a stimulus to the neuronal tissue and observing the resulting changes in blood flow correlating to that stimuli. These systems and methods have enabled improved real-time, non-contact nerve imaging.

### BRIEF DESCRIPTION OF THE INVENTION

Systems and methods are disclosed for visualizing neuronal tissue by observing the hemodynamic response of blood flow. In one embodiment of the present invention, a system is provided for evaluating the hemodynamic response of blood flow comprising: (a) a nerve stimulus means; (b) a means for capturing a pre-stimulus and a post-stimulus image of the target tissue; and (c) an image processing means for producing a processed image based on a comparison between the pre-stimulus image and the post-stimulus image.

In another embodiment, a method is provided comprising (a) acquiring a pre-stimulus image of a target tissue; (b) providing a stimulus to the target tissue; (c) introducing a time delay between the stimulus and a post-stimulus image; (d) capturing a post-stimulus image of the target tissue; and (e) producing a processed image based on a comparison between the pre-stimulus image and the post-stimulus image.

In another embodiment, a method is provided comprising (a) administering a contrast agent to the target tissue; (b) acquiring a pre-stimulus image of a target tissue; (c) providing an electrical stimulus to the target tissue; (d) introducing a delay between the electrical stimulus and a post-stimulus image of 60 to 160 milliseconds; (e) capturing the post-stimulus image of the target tissue; and (f) producing a processed image based on a comparison between the pre-stimulus image and the post-stimulus image, wherein the contrast agent may be administered to the target tissue before or after the pre-stimulus image is acquired.

### BRIEF DESCRIPTION OF THE DRAWINGS

FIG. 1 depicts an imaging system according to an exemplary embodiment of the disclosure.

FIG. 2 illustrates a real-time sequence of a stimulation-evoked hemodynamic response. Panel 2A shows a pre-stimulus image. Panel 2B shows a post-stimulus image. Panel 2C shows a subtracted image of Panels 2A and 2B.

FIG. 3 is a is a flow chart of a method for visualizing nerves of the present invention.

FIG. 4 is another flow chart of a method for visualizing nerves where a contrast agent is administered before a pre-stimulus image is acquired.

FIG. 5 is another flow chart of a method for visualizing nerves where a contrast agent is administered after a pre-stimulus image is acquired.

### RETAILED DESCRIPTION OF THE INVENTION

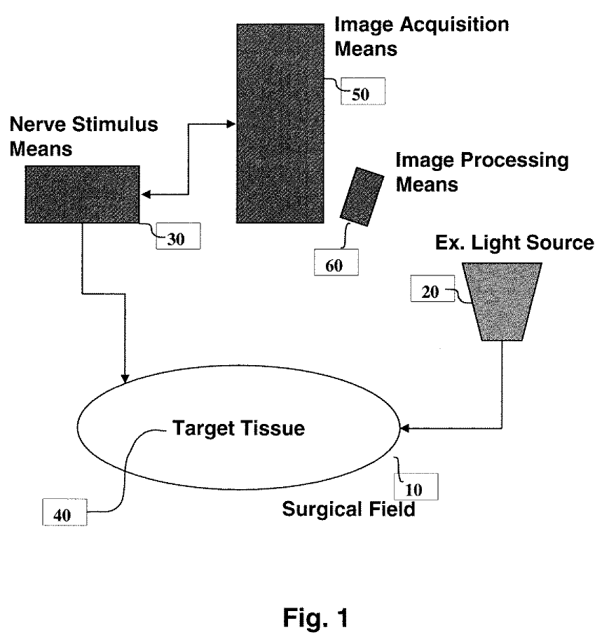

The optical imaging methods described herein employ a system comprising a nerve stimulus means; a means for capturing a pre-stimulus and a post-stimulus image of the target tissue; and an image processing means for producing a processed image based on a comparison between the pre-stimulus image and the post-stimulus image. The system may be constructed as an integrated unit, or it may be used as a collection of components. The system will be briefly described with reference to the schematic diagram, illustrated in FIG. 1, and various components and features will then be described in greater detail.

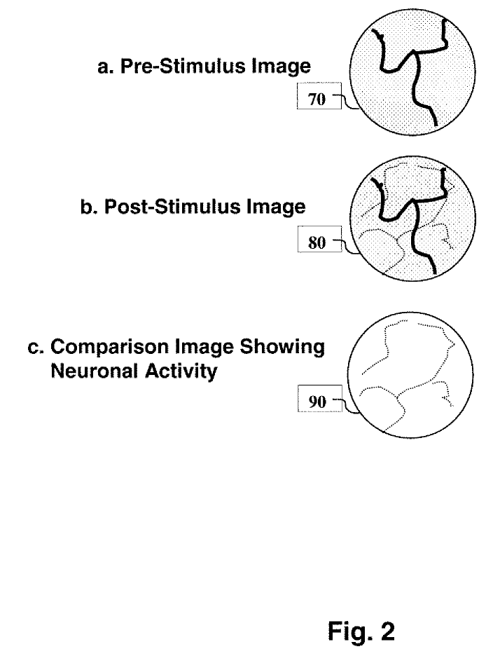

FIG. 1 illustrates a the system of the invention. As is described in greater detail below, during optical imaging the surgical field 10 is illuminated by a light source 20. A nerve stimulus means 30 is used to apply a stimulus to a target tissue 40. An image acquisition means 50 is used to obtain control data representing the pre-stimulus or “control” optical properties of an area of interest within the surgical field 10, and then to obtain subsequent data representing the post-stimulus optical properties of that area of interest, e.g., during neuronal activity. Operably connected to the image acquisition means 50 is an image processing means 60 which, as shown in FIG. 2, processes and compares the differences between one or more pre-stimulus images 70 and post-stimulus images 80 in order to derive a comparison image 90 (or images) that can be used to identify changes in optical properties representative of neuronal activity.

The surgical field 10 comprises the target tissue 40 and is the area being observed in a human, animal, etc. As used herein, the term “surgical field” refers broadly to the area in which surgical personnel are conducting a surgical procedure including but not limited to the incision site as well as any internal areas within the surgical patient that are exposed to the outside environment due to the incision. As used herein, the term “surgery” refers to any medical intervention that involves cutting or tearing the skin or other organs. In many cases, the cutting or tearing results in the exposure of internal organs and/or tissues to the environment. The invention as described herein is not limited to any particular type of surgical procedure and includes but is not limited to microscopically-aided surgery (e.g., arthroscopic surgery), as well as stereotactic and other surgical methods. As used herein, the term “surgical means” refers broadly to any item that can be used to perform or assist with surgery including but not limited to human hands, surgical instruments, lasers, robotics, remote-controlled surgical instruments, microprocessor-controlled instruments, sensors (e.g., electronic and other equipment used to assist the surgical team in assessing the status of the patient), monitors (e.g., monitors for vital function measurements), and the like. During optical imaging, the surgical field 10 is illuminated by a light source 20. The light source 20 is preferably powered by a regulated power supply. The light source 20 may be utilized to illuminate on a surgical field 10 directly, as when the target tissue 40 is exposed during or in connection with surgery, or it may be utilized to illuminate a surgical field 10 indirectly through adjacent or overlying tissue such as bone, dura, skin, muscle and the like.

As used herein, the term “light source” refers broadly to include all manner of devices that are used to produce light for industrial processes. These include lamps, lasers and other accessories that produce light anywhere along infrared spectrum. Light sources may include devices such as light emitting diodes (LED), flashlamps, light bulbs, UV lamps, filamentous light sources (with or without wavelength filtration), fluorescent lamps, incandescent lamps, tungsten halogen lamp, high intensity discharge lamps, heat lamps, spectral lamps, projection lamps, stage lamps and process UV lamps. In addition this includes, high intensity discharge lamps (HID) contain compact arc tubes, which enclose various gases and metal salts, operating at relatively high pressures and temperatures. This also includes any number of mercury lamps, metal halide lamps, sodium lamps, and xenon light sources. Laser light sources include but are not limited to ruby lasers, tunable titanium-sapphire lasers, Copper vapor lasers, a CO₂ lasers, Alexandrite lasers, argon lasers, argon-dye lasers, KTP lasers, krypton lasers, Nd:Yag lasers, xenon chloride (XeCl) excimer lasers, doubled Nd:Yag lasers, diode lasers, illuminators (e.g., backlights, LED light sources, and fiber optic illuminators), solid state lasers, helium neon lasers, nitrogen lasers, excimer lasers, ion lasers, helium cadmium lasers, laser light source pointers, and dye lasers. Additional light source types include fiber optic light sources, and deuterium light sources, as well as custom light sources for specialized applications, such as telecommunications, entertainment, art installations, medical, dental and forensic light sources.

In one embodiment, the light source 20 employed is an electromagnetic radiation (EMR) source for uniformly illuminating the surgical field 10. The EMR source may be a high intensity, broad spectrum EMR source, such as a tungsten-halogen lamp, laser, light emitting diode, or the like. Cutoff filters to selectively pass all wavelengths above or below a selected wavelength may be employed. A preferred cutoff filter excludes all wavelengths below about 695 nm. “Infrared” (IR), as used herein, refers broadly to the region of the electromagnetic spectrum bounded by the long-wavelength extreme of the visible spectrum from about 800 to 10⁶ nm. Among the bands of IR wavelengths used in the art include: near-infrared (NIR, IR-A), 700-1400 nm; short-wavelength infrared (SWIR, IR-B), 1400-3000 nm; mid-wavelength infrared (MWIR, intermediate infrared, IR-C), 3-8 μm; long-wavelength infrared (LWIR, IR-C): 8-15 μm; and far infrared: 15-1000 μm. Preferred EMR wavelengths for optical imaging include, for example, wavelengths of from about 450 nm to about 2500 nm, and most specifically, wavelengths of from about 700 nm to about 2500 nm.

Selected wavelengths of EMR may also be used, for example, when various types of contrast enhancing agents 100 are administered. The EMR source may be directed to the surgical field 10 by a fiber optic means. In one exemplary arrangement, the EMR is provided through strands of fiber optic using a beam splitter controlled by a D.C. regulated power supply (Lambda, Inc.).

It will be appreciated by those skilled in the art that the surgical field 10 and the light source 20 could be provided individually or as part of a single unit. In one embodiment, the surgical field 10 and the light source 20 are provided by an operating microscope, including but not limited to endoscopes, laparoscopes, surgical microscopes, and optical coherence tomography imaging, and others are well known in the art. One example of such a device is the fiber-optic illumination Operation Microscope OPMI 1 FC (Zeiss, West Germany).

Various types of nerve stimulus means 30 known to those of ordinary skill in the art may be used in accordance with the present invention, including an electrical stimulus, mechanical stimulus, chemical stimulus, thermal stimulus, optical stimulus, visual stimulus, or the like. Exemplary nerve stimulus means 30 commercially available for targeted nerve therapies include the NeuroTrace III (HDC Corp., Milpitas, Calif.), the Stimuplex (B.Braun America, Bethlehem, Pa.), the Digistim III euroTechnologies, Inc, Chemai, India), and the Nervonix device (Nervonix, Inc. Bozeman, Mont.), among others.

Nerve stimulation can be single, multiple, long or short impulses, or any combination of the forgoing. Stimulation may proceed, for example, over a period of 1 millisecond to more than 45 minutes, or, more specifically 1-100 seconds, or, even more specifically, 1-20 seconds. Additionally, electrical stimulation may proceed at a repetition rate, for example, of between 0.1-20 Hz, or, more specifically, 0.5-10 Hz, or, even more specifically, 1-5 Hz.

A time delay may be introduced between the stimulus 30 and a post-stimulus image 80 by any conventional means, for example, by mechanical means (e.g., a dial) or electrical means (e.g., software).

In one embodiment of invention, the system includes a target tissue 40. The target tissue 40 may be near or at the spinal column. Alternatively, the target tissue 40 may be local to the surgical site. An exemplary target tissue 40 is neural tissue. As used herein, the terms “nerves,” “neurons,” “neural tissue,” “neuronal tissue” and “nervous tissue” are used interchangeably and refer broadly to neuroanatomical structures which are enclosed, cable-like bundle of axons (including myleinated and unmyleinated nerves). Peripheral nervous system nerves include but are not limited to afferent nerves which convey sensory signals to the central nervous system (e.g., from the skin to the brain) and efferent nerves which conduct stimulatory signals from the central nervous system to the muscles and glands. In the peripheral nervous system, afferent and efferent axons are often arranged together, forming mixed nerves (e.g., the median nerve controls motor and sensory function in the hand). Central nervous system nerves include but are not limited to the twelve cranial nerves that emerge from or enter the cranium and spinal nerves which emerge from the vertebral column.

Typically the target tissue 40 is nervous tissues. The target tissue 40 may be central nervous tissue (e.g., tissue located in the brain and/or spinal cord), peripheral nervous tissue (e.g., neural tissue outside the central nervous system), somatic nervous tissue (e.g., afferent neurons that convey sensory information from the sense organs to the brain and spinal cord, and efferent neurons that carry motor instructions to the muscles), and/or autonomic nervous tissue (e.g., tissue located in the sympathetic and parasympathetic nervous systems).

In one embodiment, the target tissue 40 derives from a mammal. “Mammal” as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to alpacas, armadillos, capybaras, cats, chimpanzees, chinchillas, cattle, dogs, goats, gorillas, horses, humans, lemurs, llamas, mice, non-human primates, pigs, rats, sheep, shrews, and tapirs. Mammals include but are not limited to bovine, canine, equine, feline, murine, ovine, porcine, primate, and rodent species.

Various types of image acquisition means 50 may be used in accordance with the present invention, depending on the optical property being detected, the format of data being collected, certain properties of the area of interest, and the type of application, e.g., surgery, diagnosis, prognosis, monitoring, or the like. In general, any type of typical photon detector may be utilized as an image acquisition means 50. The image acquisition means 50 generally includes photon sensitive elements and optical elements that enhance or process the detected optical signals. Numerous optical detectors are known and may be used or adapted for use in the systems and methods of the present invention.

In one embodiment, the image acquisition means 50 is selected from the group including an optical imaging device, endoscopes, laparoscopes, surgical microscopes, and optical coherence tomography imaging, digital camera, fluorescent imaging device, ultrasound imaging device, x-ray device, MRI scanning device or a computed tomography device. The image acquisition means 50 may also include digitizing systems, such as equipment designed to convert conventional film-based x-ray images to digital data for processing and storage. In addition, the image acquisition means 50 may also be coupled to typical processing circuitry which may perform such operations as filtering, dynamic range adjustment, image enhancement, correlation between images, processing a set of images, overlaying images with data points, labeling images, saving images, changes for motion correction, and the like. The processing circuitry may be included in the image acquisition means 50, or may be part of the image processing means 60 operably connected to the image acquisition means 50.

Digital image data acquired by the image acquisition means 50 may be applied to a data storage and interface module, which may include one or more components either local to or remote from the image acquisition means 50. In one embodiment, the data storage and interface system may include local data storage, short term storage systems, archive systems, picture archiving and communications systems (PACS), and so forth. The image data may be retrievable from the data storage and interface module for processing and image enhancement in the image processing means 60, which may be operably connected to the image acquisition means 50.

Numerous image processing means 60 can be employed in the present invention. Image processing is generally operated and controlled by a host computer. The host computer may comprise any general computer (e.g., IBM PC type with an Intel, Pentium or similar microprocessor) that is interfaced with one or more of the other components of the system to direct data flow, computations, image acquisition and the like. Thus, in one embodiment the host computer controls acquisition of pre-stimulus images 70 and post-stimulus images 80 and processes those images to derive one or more comparison images 90. The host computer also preferably provides a user interface to display the comparison image(s).

Comparison images 90 may be displayed in a variety of ways. One technique for presenting and displaying comparison images 90 is in the form of visual images or photographic frames that provide a visualizable spatial location (two- or three-dimensional) of neuronal activity. In this embodiment, the comparison image 90 highlights the optical differences between the pre-stimulus image(s) 70 and the post-stimulus image(s) 80, indicative of neuronal activity. Various data processing techniques may be advantageously used to assess the comparison image 90. Processing may include averaging or otherwise combining a plurality of data sets. Data processing may also include amplification of certain signals or portions of a data set (e.g., areas of a pre-stimulus image 70 or a post-stimulus image 80) to enhance the contrast seen in the comparison images 90, and to thereby identify areas of neuronal activity and/or inactivity with a high degree of spatial resolution.

The hemodynamic response to the stimulus (or stimuli) 30 may manifest in a variety of ways, including changes in blood pressure, blood flow, blood volume, hemoglobin oxygenation, and hemoglobin concentration. In one embodiment, the hemodynamic response to the stimulus (or stimuli) 30 is an increase in total hemoglobin concentration. For example, the increase in total hemoglobin concentration may be approximately 0.1-1000% of baseline, or, more specifically, approximately 1-100% of baseline, or, even more specifically, approximately 1-10% of baseline. For each of the recited embodiments, the hemodynamic response may be observed between 1 to 1000 milliseconds, including 100, 200, 300, 400, 500, 600, 700, 800, 900, or 1,000 milliseconds, after the stimulus (or stimuli) 30 has been provided to the target tissue 40. Also, the hemodynamic response may be observed between 1 to 10 seconds, including 1, 2, 3, 4, 5, 6, 7, 8, 9, or 10 seconds, after the stimulus (or stimuli) 30 has been provided to the target tissue 40. Additionally, the hemodynamic response may be observed, more specifically, between 100 to 500 milliseconds, or, even more specifically 60 to 160 milliseconds after the stimulus (or stimuli) 30 has been provided to the target tissue 40.

In one embodiment, the system includes one or more contrast enhancing or labeling agents 100, such as dyes. For example, it may be useful to administer a contrast enhancing agent 100 to the target tissue 40 to amplify differences in an optical property being detected as a function of neuronal activity prior to acquiring subsequent data and generating a comparison. Suitable enhancing or labeling agents 100 include fluorescent agents, phosphorescent agents, luminescent agents, calorimetric agents, optical absorbing agents, quantum dots, dyes that bind to cell membranes, phase resonance dye pairs, organic fluorophores, ultrasonic contrast agents, non-fluorescent contrast agents including absorbing agents (e.g., dyes including but not limited to iso-sulfan blue, methylene blue) and scattering agents (e.g., nanoparticles), x-ray absorbing dyes, radio opaque dyes, MRI contrast agents and other well known enhancing and labeling agents. Detectors appropriate for use with such contrast enhancing agents 100 are well known in the art.

In one application, the contrast agent 100 may be provided by a slow infusion that either exravasates or remains in the vasculature to increase the sensitivity to hemodynamic changes. In another application, perfusion of the contrast agent 100 into the vasa-nervorum (i.e., the network of blood vessels supplying the nerves) may be modulated. For example, a tempanic stimulation may be applied to the nerve at the same time as administering a nerve targeting contrast agent 100 in such a way as to increase vasodilation of the vasa-nervorum for an extended period of time. In another application, a vasocontricting substance (e.g., hCGRP) may be administered to impede clearance of the contrast agent 100 from the vasa-nervorum. Should the vasodilation be conducted chemically, the contrast agent 100 and neuro-vasodilating chemical may be co-administered.

One or more components of the system may be operably connected to each another. In one embodiment, for example, the surgical field 10, light source 20, nerve stimulus means 30, image acquisition means 50 and image processing means 60 are operably connected to each other.

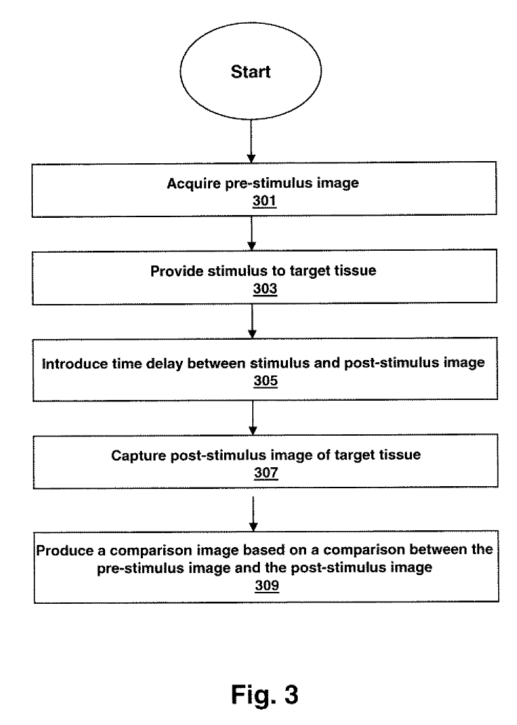

The present invention further provides a method for visualizing nerves by observing the hemodynamic response of the blood flow. An illustrative embodiment of this method is depicted schematically in FIG. 3. At block 301, a pre-stimulus image 70 (or series of images) of a target tissue 40 is acquired utilizing an image modality from the group including an optical imaging device, endoscopes, laparoscopes, surgical microscopes, and optical coherence tomography imaging, fluorescent imaging device, ultrasound imaging device, x-ray device, MRI scanning device or a computed tomography device. At block 303, a stimulus 30 selected from the group including an electrical stimulus, mechanical stimulus, chemical stimulus, thermal stimulus, optical stimulus or visual stimulus is provided to the target tissue 40. At block 305, a time delay is introduced between the stimulus 30 of the target tissue 40 and the capture of a post-stimulus image 80 (or series of images). The time delay may be introduced by mechanical means (e.g., a dial) and/or electrical means (e.g., software).

At block 307, a post-stimulus image 80 (or series of images) of the target tissue 40 is captured via an image acquisition means 50. The type of image acquisition means 50 employed typically depends on a variety of factors, including the optical property being detected, the format of data being collected, the properties of the area of interest, and the type of application, e.g., surgery, diagnosis, prognosis, monitoring, etc. In general, any type of typical photon detector may be utilized as an image acquisition means 50, including an optical imaging device, endoscopes, laparoscopes, surgical microscopes, optical coherence tomography imaging, digital camera, fluorescent imaging device, ultrasound imaging device, x-ray device, MRI scanning device or a computed tomography device or digitizing systems including equipment designed to convert conventional film-based x-ray images to digital data for processing and storage. In addition, the image acquisition means 50 may also be coupled to typical processing circuitry which may perform such operations as filtering, dynamic range adjustment, image enhancement, correlation between images, processing a set of images, overlaying images with data points, labeling images, saving images, and changes for motion correction. The processing circuitry may be included in the image acquisition means 50, or may be part of the image processing means 60 operably connected to the image acquisition means 50. Digital image data acquired by the image acquisition means 50 may be applied to a data storage and interface module, which may include one or more components either local to or remote from the image acquisition means 50. In one embodiment, the data storage and interface system may include local data storage, short term storage systems, archive systems, picture archiving and communications systems (PACS), and so forth. The image data may be retrievable from the data storage and interface module for processing and image enhancement in the image processing means 60, which may be operably connected to the image acquisition means 50.

At block 309, a comparison image 90 is produced based on a comparison between the pre-stimulus image(s) 70 and the post-stimulus image(s) 80 via an image processing means 60 generally operated and controlled by a host computer comprising any general computer (e.g., IBM PC type with an Intel, Pentium or similar microprocessor) that is interfaced with one or more of the other components of the system to direct data flow, computations, image acquisition preferably providing a user interface to display the comparison image(s). Comparison images 90 may be displayed in a variety of ways. One technique for presenting and displaying comparison images 90 is in the form of visual images or photographic frames that provide a visualizable spatial location (two- or three-dimensional) of neuronal activity. In this embodiment, the comparison image 90 highlights the optical differences between the pre-stimulus image(s) 70 and the post-stimulus image(s) 80, indicative of neuronal activity. Various data processing techniques may be advantageously used to assess the comparison image 90. Processing may include averaging or otherwise combining a plurality of data sets. Data processing may also include amplification of certain signals or portions of a data set (e.g., areas of a pre-stimulus image 70 or a post-stimulus image 80) to enhance the contrast seen in the comparison images 90, and to thereby identify areas of neuronal activity and/or inactivity with a high degree of spatial resolution.

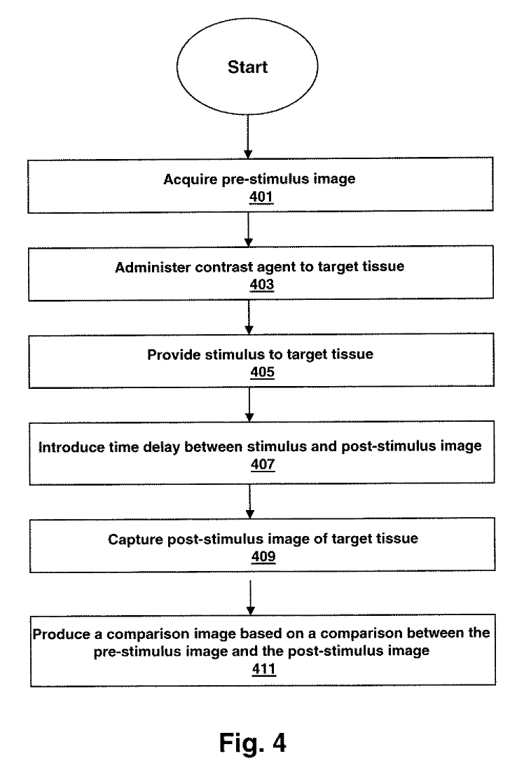

The present invention also provides a method for visualizing nerves by observing the hemodynamic response of the blood flow using a contrast agent. In one embodiment, illustrated schematically in FIG. 4, the contrast agent 100 is administered after the pre-stimulus image 70 is acquired. Referring to FIG. 4, at block 401, a pre-stimulus image 70 (or series of images) of a target tissue 40 is acquired utilizing an image modality from the group including an optical imaging device, endoscopes, laparoscopes, surgical microscopes, optical coherence tomography imaging, fluorescent imaging device, ultrasound imaging device, x-ray device, MRI scanning device or a computed tomography device. At block 403, a contrast agent 100 is administered to the target tissue 40. The contrast agent 100 may be selected from the group including dyes, fluorescent agents, phosphorescent agents, luminescent agents, colorimetric agents, optical absorbing agents, quantum dots, dyes that bind to cell membranes, phase resonance dye pairs, organic fluorophores, ultrasonic contrast agents, non-fluorescent contrast agents including absorbing agents (e.g., dyes including but not limited to iso-sulfan blue, methylene blue) and scattering agents (e.g., nanoparticles), x-ray absorbing dyes, radio opaque dyes, MRI contrast agents and other well known enhancing and labeling agents. At block 405, a stimulus 30 selected from the group including an electrical stimulus, mechanical stimulus, chemical stimulus, thermal stimulus, optical stimulus or visual stimulus is provided to the target tissue 40. At block 407, a time delay is introduced between the stimulus 30 of the target tissue 40 and the capture of a post-stimulus image 80 (or series of images). The time delay may be introduced by mechanical means (e.g., a dial) and/or electrical means (e.g., software).

At block 409, a post-stimulus image 80 (or series of images) of the target tissue 40 is captured via an image acquisition means 50. The type of image acquisition means 50 employed typically depends on a variety of factors, including the optical property being detected, the format of data being collected, the properties of the area of interest, and the type of application, e.g., surgery, diagnosis, prognosis, monitoring, etc. In general, any type of typical photon detector may be utilized as an image acquisition means 50, including an optical imaging device, endoscopes, laparoscopes, surgical microscopes, optical coherence tomography imaging, digital camera, fluorescent imaging device, ultrasound imaging device, x-ray device, MRI scanning device or a computed tomography device or digitizing systems including equipment designed to convert conventional film-based x-ray images to digital data for processing and storage. In addition, the image acquisition means 50 may also be coupled to typical processing circuitry which may perform such operations as filtering, dynamic range adjustment, image enhancement, correlation between images, processing a set of images, overlaying images with data points, labeling images, saving images, and changes for motion correction. The processing circuitry may be included in the image acquisition means 50, or may be part of the image processing means 60 operably connected to the image acquisition means 50. Digital image data acquired by the image acquisition means 50 may be applied to a data storage and interface module, which may include one or more components either local to or remote from the image acquisition means 50. In one embodiment, the data storage and interface system may include local data storage, short term storage systems, archive systems, picture archiving and communications systems (PACS), and so forth. The image data may be retrievable from the data storage and interface module for processing and image enhancement in the image processing means 60, which may be operably connected to the image acquisition means 50.

At block 411, a comparison image 90 is produced based on a comparison between the pre-stimulus image(s) 70 and the post-stimulus image(s) 80 via an image processing means 60 generally operated and controlled by a host computer comprising any general computer (e.g., IBM PC type with an Intel, Pentium or similar microprocessor) that is interfaced with one or more of the other components of the system to direct data flow, computations, image acquisition preferably providing a user interface to display the comparison image(s). Comparison images 90 may be displayed in a variety of ways. One technique for presenting and displaying comparison images 90 is in the form of visual images or photographic frames that provide a visualizable spatial location (two- or three-dimensional) of neuronal activity. In this embodiment, the comparison image 90 highlights the optical differences between the pre-stimulus image(s) 70 and the post-stimulus image(s) 80, indicative of neuronal activity. Various data processing techniques may be advantageously used to assess the comparison image 90. Processing may include averaging or otherwise combining a plurality of data sets. Data processing may also include amplification of certain signals or portions of a data set (e.g., areas of a pre-stimulus image 70 or a post-stimulus image 80) to enhance the contrast seen in the comparison images 90, and to thereby identify areas of neuronal activity and/or inactivity with a high degree of spatial resolution.

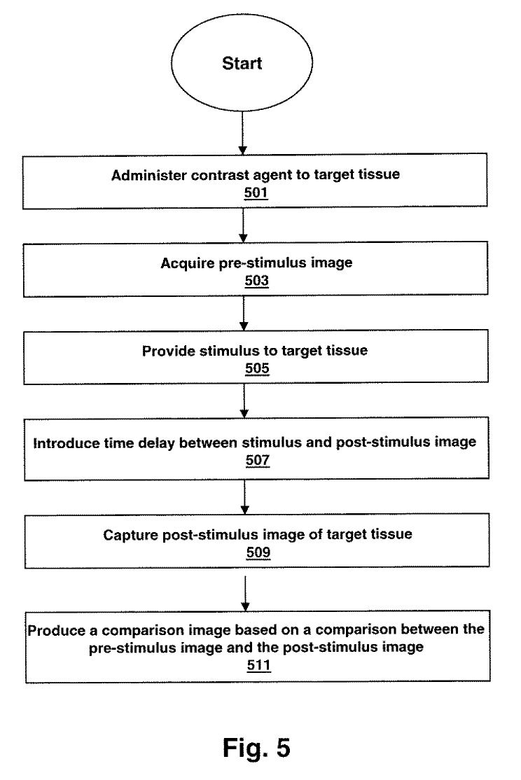

In another embodiment of the method of the invention, illustrated schematically in FIG. 5, the contrast agent 100 is administered before the pre-stimulus image 70 is acquired. Referring to FIG. 5., at block 501, a contrast agent 100 is administered to the target tissue 40 including. The contrast agent 100 may be selected from the group including dyes, fluorescent agents, phosphorescent agents, luminescent agents, calorimetric agents, optical absorbing agents, quantum dots, dyes that bind to cell membranes, phase resonance dye pairs, organic fluorophores, ultrasonic contrast agents, non-fluorescent contrast agents including absorbing agents (e.g., dyes including but not limited to iso-sulfan blue, methylene blue) and scattering agents (e.g., nanoparticles), x-ray absorbing dyes, radio opaque dyes, MRI contrast agents and other well known enhancing and labeling agents. At block 503, a pre-stimulus image 70 (or series of images) of a target tissue 40 is acquired utilizing an image modality from the group including an optical imaging device, endoscopes, laparoscopes, surgical microscopes, optical coherence tomography imaging, fluorescent imaging device, ultrasound imaging device, x-ray device, MRI scanning device or a computed tomography device. At block 505, a stimulus 30 selected from the group including an electrical stimulus, mechanical stimulus, chemical stimulus, thermal stimulus, optical stimulus or visual stimulus is provided to the target tissue 40. At block 507, a time delay is introduced between the stimulus 30 of the target tissue 40 and the capture of a post-stimulus image 80 (or series of images). The time delay may be introduced by mechanical means (e.g. a dial) and/or electrical means (e.g., software).

At block 509, a post-stimulus image 80 (or series of images) of the target tissue 40 is captured via an image acquisition means 50. The type of image acquisition means 50 employed typically depends on a variety of factors, including the optical property being detected, the format of data being collected, the properties of the area of interest, and the type of application, e.g., surgery, diagnosis, prognosis, monitoring, etc. In general, any type of typical photon detector may be utilized as an image acquisition means 50, including an optical imaging device, endoscopes, laparoscopes, surgical microscopes, optical coherence tomography imaging, digital camera, fluorescent imaging device, ultrasound imaging device, x-ray device, MRI scanning device or a computed tomography device or digitizing systems including equipment designed to convert conventional film-based x-ray images to digital data for processing and storage. In addition, the image acquisition means 50 may also be coupled to typical processing circuitry which may perform such operations as filtering, dynamic range adjustment, image enhancement, correlation between images, processing a set of images, overlaying images with data points, labeling images, saving images, and changes for motion correction. The processing circuitry may be included in the image acquisition means 50, or may be part of the image processing means 60 operably connected to the image acquisition means 50. Digital image data acquired by the image acquisition means 50 may be applied to a data storage and interface module, which may include one or more components either local to or remote from the image acquisition means 50. In one embodiment, the data storage and interface system may include local data storage, short term storage systems, archive systems, picture archiving and communications systems (PACS), and so forth. The image data may be retrievable from the data storage and interface module for processing and image enhancement in the image processing means 60, which may be operably connected to the image acquisition means 50.

At block 511, a comparison image 90 is produced based on a comparison between the pre-stimulus image(s) 70 and the post-stimulus image(s) 80 via an image processing means 60 generally operated and controlled by a host computer comprising any general computer (e.g., IBM PC type with an Intel, Pentium or similar microprocessor) that is interfaced with one or more of the other components of the system to direct data flow, computations, image acquisition preferably providing a user interface to display the comparison image(s). Comparison images 90 may be displayed in a variety of ways. One technique for presenting and displaying comparison images 90 is in the form of visual images or photographic frames that provide a visualizable spatial location (two- or three-dimensional) of neuronal activity. In this embodiment, the comparison image 90 highlights the optical differences between the pre-stimulus image(s) 70 and the post-stimulus image(s) 80, indicative of neuronal activity. Various data processing techniques may be advantageously used to assess the comparison image 90. Processing may include averaging or otherwise combining a plurality of data sets. Data processing may also include amplification of certain signals or portions of a data set (e.g., areas of a pre-stimulus image 70 or a post-stimulus image 80) to enhance the contrast seen in the comparison images 90, and to thereby identify areas of neuronal activity and/or inactivity with a high degree of spatial resolution.

Any of the steps of the method of the invention may be repeated to improve image quality. For example, a series of pre-stimulus and/or pro-stimulus images can be captured and processed.

The systems and methods described herein can be used to visualize nerves during surgical or diagnostic procedures and to monitor neuronal activity and/or inactivity. For example, the systems and methods can be used by a surgeon intraoperatively to distinguish between neuronal tissue and surrounding non-neuronal tissue.

The systems and methods described herein can be used to identify and locate individual nerves for diagnostic purposes (e.g., biopsy) or to avoid damaging nerves during surgery. Numerous surgical procedures involve potential nerve damage, including for example, procedures involving veins, glands (e.g., thyroid and prostate gland), operations on the hand (e.g., carpel tunnel syndrome), operations in the urogential area (e.g., gynecological operations). The systems and methods described herein can also be used to identify and locate individual nerves, for example, during neurosurgical procedures involving anastomoses of severed nerves or during other types of surgery involving peripheral tissue, enabling the surgeon to avoid damage to nerves.

These systems and methods can be used to provide information in “real time” and therefore can be employed intraoperatively. These systems and methods can also be used over a more prolonged period, such as during monitoring of neuronal tissue viability, trauma, recovery, and the like.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A system for evaluating the hemodynamic response of blood flow in a target tissue comprising:
a) a nerve stimulus means;
b) an image acquisition means for capturing a pre-stimulus and a post-stimulus image of the target tissue; and
c) an image processing means for producing a processed image based on a comparison between the pre-stimulus image and the post-stimulus image.

2. The system of claim 1, wherein the nerve stimulus means is selected from the group consisting of an electrical stimulus, mechanical stimulus, chemical stimulus, thermal stimulus, optical stimulus, visual stimulus, or any combination thereof.

3. The system of claim 2, wherein the nerve stimulus means is an electrical stimulus.

4. The system of claim 1, wherein the image acquisition means is selected from the group consisting of an optical imaging device, endoscopes, laparoscopes, surgical microscopes, optical coherence tomography imaging, fluorescent imaging device, ultrasound imaging device, x-ray device, MRI scanning device, a computed tomography device, or any combination thereof.

5. The system of claim 4, wherein the image acquisition means is a fluorescent imaging device.

6. The system of claim 1, wherein the system further comprises a target tissue.

7. The system of claim 1, wherein the target tissue is located in the central nervous system or peripheral nervous system.

8. The system of claim 7, wherein the central nervous system nerves are cranial nerves.

9. The system of claim 7, wherein the peripheral nervous system nerves are afferent nerves or an efferent nerves.

10. A method of visualizing nerves by observing the hemodynamic response of the blood flow, comprising:
a) acquiring a pre-stimulus image of a target tissue;
b) providing a stimulus to the target tissue;
c) introducing a time delay between the stimulus and a post-stimulus image;
d) capturing a post-stimulus image of the target tissue; and
e) producing a processed image based on a comparison between the pre-stimulus image and the post-stimulus image.

11. The method of claim 10, wherein acquiring the pre-stimulation image further comprises utilizing an image modality selected from the group consisting of an optical imaging device, endoscopes, laparoscopes, surgical microscopes, optical coherence tomography imaging, fluorescent imaging device, ultrasound imaging device, x-ray device, MRI scanning device, a computed tomography device, or any combination thereof.

12. The method of claim 10, further comprising administering a contrast agent to the target tissue.

13. The method of claim 12, wherein the contrast agent is selected from the group consisting of a fluorescent agent, optical absorbing agent, an ultrasonic contrast agent, or any combination thereof.

14. The method of claim 13, wherein the contrast agent is a fluorescent agent.

15. The method of claim 10, wherein the stimulus is selected from the group consisting of an electrical stimulus, mechanical stimulus, chemical stimulus, thermal stimulus, optical stimulus, visual stimulus, or any combination thereof.

16. The method of claim 15, wherein the stimulus an electrical stimulus.

17. The method of claim 10, wherein the nerves are in the central nervous system or peripheral nervous system.

18. The method of claim 17, wherein the central nervous system nerves are cranial nerves.

19. A method of visualizing nerves by observing the hemodynamic response of the blood flow, comprising:
a) administering a contrast agent to a target tissue;
b) acquiring a pre-stimulus image of a target tissue;
c) providing an electrical stimulus to the target tissue;
d) introducing a time delay between the electrical stimulus and a post-stimulus image;
e) capturing the post-stimulus image of the nerve; and
f) producing a processed image based on a comparison between the pre-stimulus image and the post-stimulus image,
wherein the contrast agent may be administered to the target tissue before or after the pre-stimulus image is acquired.

20. The method of claim 19, wherein said time delay between the electrical stimulus and a post-stimulus image is about 60 to 160 milliseconds.
